(19) United States
(12) Patent Application Publication
(10) Pub. No.: US 2008/0234362 A1
(43) Pub. Date: Sep. 25, 2008

(54) Treatment for Asthma and Arthritis and Other Inflammatory Diseases

(75) Inventor: Anthony Michael Chandler, Surrey (GB)

Correspondence Address: CONLEY ROSE, P.C.;David A. Rose, P. O. BOX 3267, HOUSTON, TX, 77253-3267, US

(73) Assignee: BIONOVATE LIMITED, Cambridgeshire (GB)

(21) Appl. No.: 11/569,212

(22) PCT Filed: May 17, 2005
(86) PCT No.: PCT/GB05/01885

§ 371 Date: Apr. 20, 2007

(30) Foreign Application Priority Data
May 19, 2004 (GB) 0411166.2

(51) Int. Cl. A61K 31/352; A61P 11/06; A61P 9/00; A61P 19/02

(52) U.S. Cl. 514/456

(57) ABSTRACT

A synergistic effect is obtained in the treatment of combined omega-3 series polyunsaturated fatty acids and flavonoids in the treatment of asthma, chronic obstructive pulmonary disease, rheumatoid and osteoarthritis, and other inflammatory conditions. The fatty acids are extracted from the New Zealand Green Lipped Mussel Perna canaliculus.

This invention makes use of the synergistic effect of combined omega-3 series polyunsaturated fatty acids and flavonoids (also referred to as bioflavonoids) upon asthma, chronic obstructive pulmonary disease and rheumatoid and osteoarthritis, and other inflammatory conditions.

Mammalian inflammatory pathways are an important consequence of the immune system and play a vital role in the normal homeostasis of the body. Whilst short-term inflammation has a protective function, in chronic diseases such as arthritis and asthma, inflammation is associated with the typical oedema, swelling, pain and organ dysfunction.

Arthritis and asthma are major chronic diseases worldwide that produce an enormous socioeconomic burden. Asthma has an allergic component in its aetiology and the incidence of asthma is set to double within 15 years providing a continued challenge to long term therapeutic control. Arthritis continues to be of considerable impact to the lives of millions and is believed to affect 15% of the population in its chronic form.

The use of Polyunsaturated Fatty Acids (PUFAs) such as the omega-3 and omega-6 series in the amelioration of inflammation in both arthritics and asthmatics has been well documented. PUFAs influence the mammalian inflammatory pathways due to their interaction with the metabolism and supply of arachidonic acid into the cyclo-oxygenase and lipoxygenase enzyme pathways that produce potent prostaglandins and leukotrienes respectively.

Prostaglandins and leukotrienes are potent biologically active structures that normally play an essential role in tissue homeostasis. However, following cellular injury or trauma the respective production of specific prostaglandins and leukotrienes shifts to an inflammatory reaction with local physiological effects [see Table 1].

What is perhaps to some extent less widely appreciated is the structural similarities exhibited by these essential physiological mediators and in particular their shared metabolic precursor, arachidonic acid. Arachidonic acid, prostaglandins and leukotrienes are PUFA structures with a 20-carbon chain and are therefore described as Eicosanoids. They are synthesised in almost every tissue but are not stored in any significant quantities. These eicosanoid PUFAs therefore act as the precursor to the arachidonic acid cascade.

TABLE 1
Source and physiological response produced by some of the products of the
arachidonic acid cascade.

| Eicosanoid | Primary source | Physiologic response |
|---|---|---|
| Prostaglandin D₂ | Mast cell, | Vasodilation, bronchoconstriction |
| (PGD₂) | multiple other | |
| | tissues | |
| Prostaglandin | Multiple tissues | Vasoconstriction, uterine and bronchial smooth |
| F₂ₐₗₚₕₐ (PGF₂ₐₗₚₕₐ₎ | | muscle contraction |
| Prostacyclin | Vascular | Vasodilation, inhibits platelet aggregation, acute |
| (PGI₂) | endothelium, | inflammatory reactions |
| | macrophages | |
| Thromboxane A₂ | Platelets, white | Vasoconstriction, platelet aggregation |
| (TXA₂) | blood cells | |
| Prostaglandin E₂ | White blood cells, | Vasodilation, acute inflammatory response, |
| (PGE₂) | multiple other | inhibits gastric acid secretion, pyrexia, |
| | tissues | analgesia, inhibits renal tubular reabsorption, |
| | | stimulates osteoclastic activity |

In the following description of the invention and the background thereto, reference will be made to the accompanying drawings, in which:

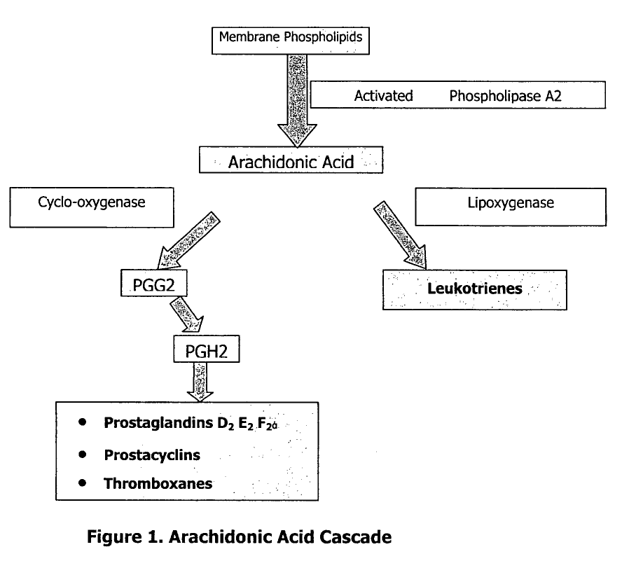

FIG. 1: shows the Arachidonic Acid Cascade;

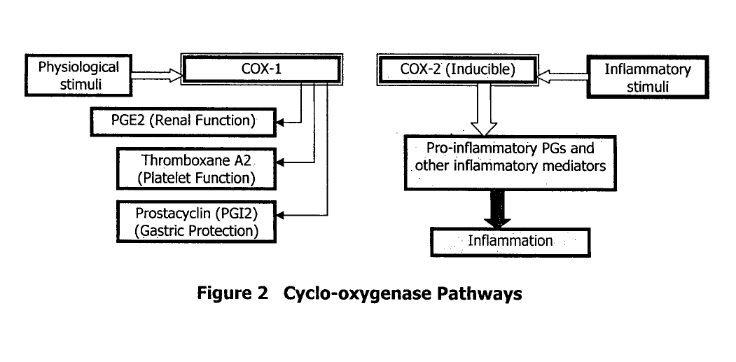

FIG. 2: shows the to cyclo-oxygenase pathways; and

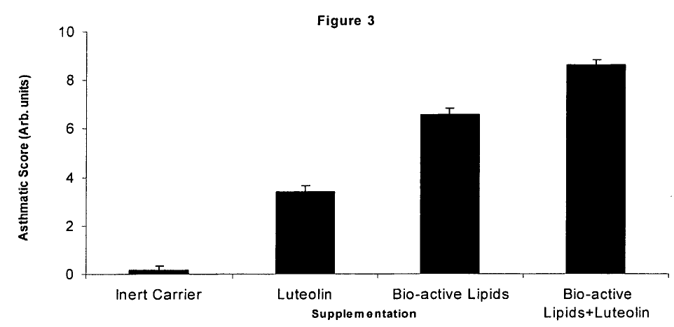

FIG. 3: shows results obtained in Example 2.

### EICOSANOID METABOLISM

Eicosanoids are 20-carbon compounds derived from polyunsaturated fatty acids, also known as the eicosanoic acids and which serve as precursors to a variety of other biologically active compounds within cells. These include prostaglandins, thromboxanes and leukotrienes, which are themselves eicosanoids and are therefore based upon the eicosanoid 20-carbon structure.

At the cellular level, arachidonic acid is one of the major sources of 20-carbon structures which provide the essential precursors of prostaglandins (sometimes referred to as Prostanoids), thromboxanes and leukotrienes. These compounds act as biological regulators within animals and their function depends upon the type of tissue and relevant enzyme systems involved and are well known mediators of inflammation and immune response.

Eicosanoid metabolism is controlled by the availability of arachidonic acid or other eicosanoid structures, enzyme expression and negative or positive feedback loops for example. Eicosanoids are potent regulators of cell metabolism but have a short half-life of less than 5 minutes allowing for significant control over physiological functions. Their potency is such that the ratio of body mass to eicosanoid mass is in the order of 1 million.

In recent years pharmacological research has begun to unravel the complexities of mammalian inflammatory pathways leading to increased pharmaceutical interest in novel compounds that can provide anti-inflammatory activity with reduced adverse effects, contra-indications or toxicity.

### Eicosanoids and the Inflammatory Process

The inflammatory process begins with cell injury. Trauma, infection, or other injury to the cell which activates membrane bound phospholipase A2 (pLA2), which releases arachidonic acid from the injured cell's membrane. Arachidonic acid fuels the cyclo-oxygenase and lipoxygenase inflammatory pathways.

The inflammatory process directly involves eicosanoid metabolism. Of the numerous mechanisms involved a number of pathways are of particular interest, the cyclo-oxygenase (or COX) and lipoxygenase (LOX) pathways, both of which constitute the Arachidonic Acid Cascade shown in FIG. 1.

The arachidonic acid cascade is responsible for the production of various biological regulators at the tissue level. Control of eicosanoid metabolism can be achieved by the supply of arachidonic acid, negative feedback mechanisms and therapeutically by treatment with non-steroidal anti-inflammatory drugs (NSAIDs) for example.

The biochemical by-products of this process have been implicated in many divergent physiologic responses to inflammation: vasodilation, bronchoconstriction, vasoconstriction, smooth muscle contraction, platelet aggregation, pyrexia, analgesia, inhibition of renal tubular sodium reabsorption, stimulation of osteoclastic activity and inhibition of gastric acid secretion (see Table 1).

### The Lipoxygenase Pathway

Lipoxygenase is an enzyme that converts arachidonic acid to several intermediates, including 5-hydroperoxyeicosatetraenoic acid (5-HPETE), which gives rise to the leukotrienes (LTA4, LTB4, LTC4, and LTD4). Leukotrienes play a role in vascular permeability and they are potent chemotactic factors, increasing White Blood Cell (WBC) migration into inflamed tissues. Leukotrienes are associated with the development of oedema and WBC effusion into tissues such as joints and lung endothelium in arthritis and asthma respectively. Recently a number of anti-leukotriene therapies have been licensed for the treatment of asthma Most research has concentrated on NSAIDs demonstrating varying efficacies. The widely varying profiles of currently available NSAIDs may be explained by the discovery of two isoforms of the cyclo-oxygenase enzyme possessing different profiles, see FIG. 2.

Cyclo-oxygenase 1 (COX 1) has a physiological role and influences the normal activities of platelet aggregation, gastric mucosa, and kidney. COX1 activity is not influenced by inflammatory stimulation.

Cyclo-oxygenase 2 (COX 2) is induced by inflammatory stimulation releasing pro-inflammatory prostaglandins.

The increased production of prostaglandins accompanying the arachidonic acid cascade is regulated by the supply of arachidonic acid. The inflammatory reaction is therefore a two stage process; increased enzyme expression and increased arachidonic acid supply.

Thus it follows that the inflammatory reaction is dependent upon the availability of supply of arachidonic acid. It also follows that the inflammatory process can be influenced by the manipulation of the arachidonic acid concentration and therefore is dependent upon the availability of PUFAs.

Arachidonic acid production and availability at the cell membrane depends upon dietary intake of essential fatty acids such as omega-6 linoleic acid. Its release from the cell membrane by phospholipase A2 clearly can influence the availability of this vital eicosanoid precursor at the active site of COX and LOX enzymes.

### Naturally Occurring Eicosanoids and the Role of PUFAs

The most recognised naturally occurring eicosanoids are found in marine-derived oils such as fish oils which contain the omega-3 series of Polyunsaturated Fatty Acids (PUFAs). Fish oil is a well known source of one such eicosanoid in particular, namely eicosapentaenoic acid or EPA. EPA has been used for many years with little, if any, evidence of clinical anti-inflammatory activity at the dose commonly used.

PUFAs are not only required for energy, but are implicated in the regulation of biochemical pathways within the body. In particular, PUFAs are the obligate precursors of a wide range of signalling molecules, including the prostanoids, which have a central role in inflammatory responses. Thus altering dietary PUFA composition may have a considerable influence on the inflammatory response through alterations in the type and relative quantities of prostanoids synthesised.

In general, the 2-series prostaglandins (derived from n-6 PUFAs) are far more pro-inflammatory than the 3-series prostaglandins (derived from n-3 PUFAs), so increases in the proportion of n-3 PUFA precursors in the body should have significant anti-inflammatory effects. The benefits of this are far-reaching as a means for minimising respiratory disease and arthritis, concomitant with reduced need for drug intervention.

Further results have shown that n-3 PUFAs inhibit the conversion of the precursor lipid, arachidonic acid, by the lipoxygenase and cyclo-oxygenase pathways to pro-inflammatory metabolites such as leukotriene B4 (LTB4), 5-hydroxyeicosopentaenoic acid (HETE), and thromboxane A2. The leukotrienes. LTC₄, LTD₄ and LTE₄ have been shown to produce strong bronchospastic responses in central and peripheral airways and reduce airflow dramatically in asthma, adult respiratory distress syndrome, hypoxic pulmonary hypertension and LPS-induced pulmonary injury. The n-3-PUFA linolenic acid has been shown to reduce leukotriene production in adult asthmatics.

It has been demonstrated in knock-out mice, that a deficiency of PGHS-1 and PGHS-2 (the key prostaglandin synthetic enzymes), greatly reduces the inflammatory response in allergic lung responses. These studies confirm the importance of arachidonic acid metabolites in responses to respiratory challenges. Whilst a certain level of eicosanoids is required for ‘housekeeping’ purposes and the establishment of an immune response is a necessary function, the exact quantities and type of prostanoid synthesised may be crucially altered by an imbalance of n-3/n-6 PUFAs resulting in physiological systems such as the pulmonary airways and joints becoming hyper-sensitive to harmful environments and infection. The advantages of using n-3 PUFAs to inhibit arachidonic acid metabolism is that, unlike most commonly used anti-inflammatory drugs, they do not completely block cyclo-oxygenase activity, thus allowing for synthesis of beneficial prostanoids such as prostacyclin and PGE₂.

### The use of Anti-Inflammatory Omega-3 Series Polyunsaturated Fatty Acids in the Management and Treatment of Asthma.

It is has been well known for some time that changes in dietary fatty acids can modulate inflammatory activity, see for example Leaf A and Weber PC. N Engl J Med 1988;318:549-57. Differences in fatty acid intake translates into differences in the fatty acid content of lipid membranes and other substrates, which are in turn the substrates for eicosanoid production (Goodnight S H Jr, et al, Arteriosclerosis 1982;2:87-113).

Therefore, changes in the substrates can alter the distribution of the eicosanoids produced in the body. In particular the presence of omega-3 PUFAs lowers the production of inflammatory eicosanoids through:

Competition with arachidonic acid as a constituent of lipid membranes
Competition with arachidonic acid as a substrate for prostaglandin endoperoxide synthase (cyclo-oxygenase) activity
Inhibition of the conversion of linoleic acid to arachidonic acid
Reduced production of inflammatory leukotrienes in the lipoxygenase pathway

Eicosapentaenoic Acid (EPA) (C20:5n3), docosahexaenoic acid (DHA) (C22:6n3) and a-linolenic acid (aLNA) (C18:3n3) are the most widely researched omega-3 PUFAs and have been variously reported to benefit anti-inflammatory conditions. With reference to asthma, results have been described as controversial with some evidence indicating a positive effect upon the symptoms of asthma and some against. The reason for this may be due to the proportion and type of omega-3 series PUFA used and dosage given.

The parent omega-3 series PUFA is a-linolenic acid, which is the key dietary source of omega-3 PUFAs from which EPA is derived. At levels of 9 g/day, a-LNA derived from Perilla oil has been shown to inhibit LTB4 production and improve pulmonary function in asthmatic patients, (Okamoto M et al, Intern Med 2000;39(2):107-11). EPA acts as a competitive membrane-bound PUFA to arachidonic acid. At normal intake levels of a-LNA, whether by food or supplement usage, neutrophil function is modulated but clinical efficacy is small or not significant. Dosages higher than 3.2 g per day are required to demonstrate significant reductions in typical asthma scores (Arm J P et al, Thorax, 1988;43:84-92).

Pharmacological Application of Lipid-Derived Omega-3 Series Poly-Unsaturated Fatty Acids from Perna Canaliculus

The anti-arthritic properties of the New Zealand Green Lipped Mussel (Perna canaliculus) have been reviewed for nearly 30 years. More recently the range of omega-3 series PUFAs naturally present in Perna canaliculus have been evaluated for their anti-inflammatory and anti-asthmatic properties. These marine-derived lipids have been shown to possess potent anti-inflammatory properties by inhibiting the action of the two enzymes, cyclo-oxygenase and lipoxygenase.

U.S. Pat. No. 63,462,278 describes a method of anti-inflammatory treatment of a human or animal patient comprising administration of a lipid extract of Perna canaliculus. U.S. Pat. No. 6,596,303 describes the alleviation of arthritic symptoms in animals by administering powdered Perna canaliculus in the feed. WO03043570A2 describes formulations and methods of treatment of inflammatory conditions comprising an omega-3 fatty acid, such as DHA, or a flavonoid with a non-alpha tocopherol. WO03011873A2 describes a phospholipid extract from a marine biomass comprising a variety of phospholipids, fatty acid, metals and a novel flavonoid. WO02092450A1 describes the production and use of polar rich fractions containing EPA, DHA, AA, ETA and DPA from marine organisms and others and their use in humans food, animal feed, pharmaceutical and cosmetic applications.

The lipids extracted from the Green Lipped Mussel have been shown to contain particular types of fatty acids not found in the same proportion in other organisms. These omega-3 series PUFAs have only recently been characterized due to advances in manufacturing. It is essential that cold processing and suitable drying methods are used to preserve the delicate structures of these particular fatty acids. The omega-3 series content is known to include the PUFAs: EPA, DHA and the ETAs (eicosatetraenoic acids).

The ETAs have a similar structure to the omega-6 series arachidonic acid but have been shown to be profoundly more potent than EPA, DHA or a-LNA in inhibiting the production of proinflammatory prostaglandins, thromboxanes and leukotrienes. ETAs have been shown to be as potent as ibuprofen and aspirin in independent studies and 200 times more potent than EPA in the rat paw oedema test (Whitehouse M W et al, Inflammopharmacology 1997; 5:237-246).

Pharmacologically, lipid derived from Perna canaliculus has been shown to significantly inhibit cyclo-oxygenase 2 and Lipoxygenase pathways following in vitro studies that determined the IC50 for each:
Cyclo-oxygenase 2 IC50=1.2 μg/ml
Lipoxygenase IC50=20 to 50 ug/ml

Therefore, the lipids occurring naturally in Perna canaliculus exhibit significant anti-inflammatory activity in vitro and in vivo

Anti-Asthmatic Effect of Lipids Derived from Perna Canaliculus

Studies utilising an omega-3 series lipid extract from Perna canaliculus containing ETAs have produced significant results in mild asthma sufferers after 8-weeks treatment at a dosage of 50 to 200 mg/day (Emelyanov A et al, Eur. Respir. J 2002;20:596-600). Significant beneficial effects have been seen in:—
Daytime wheeze
Reduced B₂ agonists
Morning peak expiratory flow (PEF)
Exhaled H₂O₂
   Steroid Sparing Effect of Lipids Derived from Perna Canaliculus

An asthmatic patient using the Perna canaliculus-derived lipids for anti-arthritic effects retrospectively noted an 80% reduction in the use of prednisone from 167 mg/month to 31 mg/month. (Harbison S J and Whitehouse, M W, Medical Journal of Australia, 200;173:560).

The lipids from Perna are relatively slow acting but have significant beneficial effects in mild cases of asthma where they may have a disease modifying action.

### The use of Bioflavonoids in the Management of Asthma

Flavonoids constitute an important group of dietary polyphenols, which are widely distributed in plants. Over 4000 different flavonoids have been described, and they are categorized into flavonols, flavones, flavanones, anthocyanidins, and isoflavones.

Rutin has been proposed in U.S. Pat. No. 6,326,031 for use in a composition intended to combat cardiovascular diseases which further includes fish oil as a source of EPA and DHA, capsaicin and garlic powder, made up as a food supplement. The intended role of rutin in this composition is unclear.

Particularly rich dietary sources of flavonoids are red grape juice, red wine, green and black tea, cocoa and chocolate, various fruits, green vegetables and onions.

Some of the flavonols occur as covalently linked oligomers, the procyanidins. Although the flavonoids do not belong to the vitamins, their daily intake is in the same order of magnitude of that of the antioxidant vitamins C and E. Therefore they are classified as micronutrients.

The flavonoids and other dietary polyphenols contribute to the antioxidant defence system of the organism against oxidative stress.

Flavonoids have also been reported to exert anticancer and antimicrobial activities. A number of in vitro and in vivo studies as well as clinical trials suggest beneficial effects of flavonoids for health. In particular, high intake of flavonoids is believed to counteract the development of cardiovascular diseases.

Flavonoids have demonstrated a variety of biological effects including anti-oxidation, anti-inflammation, anti-allergic effects, anti-platelet, and anti-thrombotic actions.

For example, an in vitro oxidation model showed quercetin, myricetin, and rutin are more efficient antioxidants than traditional vitamins. Some flavonoids, especially quercetin, protect low-density lipoprotein from oxidative damage in vitro and are thought capable of reducing the risk of coronary heart disease or cancer. Flavonols and flavones also have antioxidant and free radical scavenging activity in foods. Epidemiological studies have indicated a relationship between a diet rich in flavonols and a reduced incidence of heart disease. Others, such as the anthocyanidins from some purple plant foods may help protect the lens of the eye. Soy isoflavones are also currently being studied to see if they help fight cancer. Quercetin has been reported to block the “sorbitol pathway” which is linked to many problems associated with diabetes. Rutin and several other flavonoids may also protect blood vessels.

Their mode of antioxidant action appears to be multivalent and occurs at three different levels: (i) scavenging of free radicals and reactive oxygen and nitrogen species, (ii) chelating of transition metal ions, thus masking the pro-oxidant actions, (iii) ameliorating deleterious actions of pro-oxidant enzymes (lipoxygenases, myeloperoxidase and others).

The USDA Database for the Flavonoid Content of Selected Foods, released in March 2003, contains information on the most prevalent dietary flavonoids. These are organized into five subclasses based on their chemical structure:

### Flavonols

Quercetin, Rutin (a glycosylated form of quercetin), Kaempferol, Myricetin, Isorhamnetin,

### Flavones

Apigenin, Luteolin

### Flavanones

Hesperetin, Naringenin, Eriodictyol

### Flavan-3-Ols

Catechins, Epicatechins, Theaflavins, Thearubigins

### Anthocyanidins

Cyanidin, Delphinidin, Malvidin, Pelargonidin, Peonidin, Petunidin

The flavonoids are components of many common vegetables. For instance, the flavones (the group containing luteolin) are found in celery green hearts, celery, parsley and rutabagas and other sources.

### Comparative Anti-TNF and Anti-Inflammatory Activity

Many bioflavonoids have been determined to demonstrate anti-inflammatory activity in vitro.

However, it is important to demonstrate efficacy both in vivo and in vitro and there may be different comparative results between flavonoids and in some cases different activity has been found.

The difference between the in vitro and in vivo activities has been attributed to the relative positions of hydroxyl groups in the chemical structure. In a study of flavonoids, potent oral in vivo anti-TNFα activity was only found in luteolin and apigenin. It is therefore apparent that a particular hydroxylated structure common to apigenin and luteolin is required for the anti-TNFα activity demonstrated orally. In this case luteolin was more potent than apigenin. (Biosci. Biotechnol. Biochem 2004 68 (1) 119-125).

Tumour necrosis factor (TNFα) is a pleiotropic multifunctional cytokine and a central regulator of inflammatory processes. TNFα has been implicated in a number of diseases including asthma, rheumatoid arthritis, multiple sclerosis and other inflammatory disorders. It is implicated in cell death and apoptosis but it is able also to generate a non-cytotoxic inflammatory response in certain situations. It has been shown conclusively to be released immediately from mast cells after encounter with specific allergens and is therefore implicated in allergic asthma. Like other cytokines, TNFα confers its signals to target cells through binding to distinct membrane receptors, referred to as p55 or TNFR1 and p75 or TNFR2. Although FR1 is the major biologically active receptor, both receptors exert unique activities. TNFα is a likely central mediator of airway inflammation and bronchial hyper-responsiveness in asthma. It is measured at high levels in bronchoalveolar lavage fluid (BALF) and can regulate inflammatory cell infiltration, locally enhance vascular permeability and aid in the release of bronchoactive substances such as histamine. Among other functions, TNFα promotes the migration of dendritic cells, part of a family of antigen-presenting cells present in many organs.

In terms of oral anti-inflammatory activity using the established TPA ear oedema test, only luteolin and quercetin were effective (Biol. Pharm. Bull 2002 25(9) 1197-1202). Thus, oral anti-inflammatory activity requires a structure common to quercetin and luteolin, which is 3′,4′,5′,7-tetrahydroxyflavone. Only luteolin inhibited both serum TNFα production and TPA-induced ear oedema. Luteolin therefore has an optimal structure to inhibit allergic inflammation in a number of ways.

### Studies of Structure Activity Relationships of Flavonoids for Anti-Allergic Activity

Further studies of the relationship of structural conformation to anti-allergic activity has been evaluated. The IC₅₀ values for the degranulation of mast cells was determined for 22 flavonoid compounds. The results are shown in the table below (Arch. Pharma Res. 1998 21(4) 478-480)

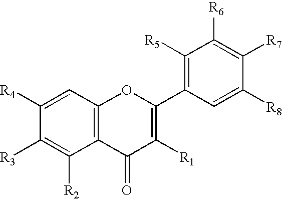

| Name | R₁ | R₂ | R₃ | R₄ | R₅ | R₆ | R₇ | R₈ | IC₅₀ (μM) |
|---|---|---|---|---|---|---|---|---|---|
| Flavone | —H | —H | —H | —H | —H | —H | —H | —H | >100.0 |
| 3-hydroxyflavone | —OH | —H | —H | —H | —H | —H | —H | —H | >100.0 |
| 6-hydroxyflavone | —H | —H | —OH | —H | —H | —H | —H | —H | 28.0 |
| 7-hydroxyflavone | —H | —H | —H | —OH | —H | —H | —H | —H | 21.0 |
| Chrysin | —H | —OH | —H | —OH | —H | —H | —H | —H | >100.0 |
| Baicalein | —H | —OH | —OH | —OH | —H | —H | —H | —H | 17.0 |
| Apigenin | —H | —OH | —H | —OH | —H | —H | —OH | —H | 4.5 |
| Luteolin | —H | —OH | —H | —OH | —H | —OH | —OH | —H | 1.8 |
| Diosmetin | —H | —OH | —H | —OH | —H | —OH | —OCH₃ | —H | |
| 3,6-dihydroxyflavone | —H | —OH | —H | —OH | —H | —H | —H | —H | 6.0 |
| Diosmin | —H | —OH | —H | —OS* | —OH | —H | —OCH₃ | —H | >100 |
| Fisetin | —OH | —H | —H | —OH | —H | —OH | —OH | —H | 3.3 |
| Galangin | —OH | —OH | —H | —OH | —H | —H | —H | —H | 40.0 |
| Kaempferol | —OH | —OH | —H | —OH | —H | —H | —OH | —H | 7.5 |
| Quercetin | —OH | —OH | —H | —OH | —H | —OH | —OH | —H | 3.0 |
| Myricetin | —OH | —OH | —H | —OH | —H | —OH | —OH | —OH | 6.7 |
| Morin | —OH | —OH | —H | —OH | —OH | —H | —OH | —H | 51.0 |

*represents sugar.

A number of flavonoids demonstrated potent activity in this anti-allergy model confirming the general efficacy of the flavonoids structure. The most active were luteolin, apigenin, diosmetin, fisetin and quercin. Luteolin was confirmed to be the most potent.

### Inhibition of IgE-Mediated Allergic Reactions and TNFα by Flavonoids

The comparative action of the flavonoids baicalein, quercetin and luteolin have been investigated in a mouse and rat model assessing the production of histamine and other cytokines such as TNFα and IL-1B induced by IgE. They were found them to be more potent than many other agents investigated including therapeutics and immunosuppressors.

Luteolin and other flavonoids have a significant effect upon mast cell responses to IgE induced allergic responses inhibiting histamine, TNFα and IL-1. All three flavonoids were effective in inhibiting histamine release from human cultured mast cell (HCMC) using two histamine stimulants.

### Luteolin is Effective in Reducing Asthma Symptoms

Airway conductance and hyper-responsiveness are key symptoms diagnostic of asthma. Using ovalbumin as an allergen, mice were sensitised and then exposed to allergen, with luteolin being given orally before (pre-sensitisation) and curatively after sensitisation. Airway conductivity and hyper-responsiveness were assessed. Luteolin significantly reduced antigen-induced bronchoconstriction and airway hyper-reactivity when given orally before and after sensitisation. (Inflamm Res. 2003 March;52(3):101-106).

### Percutaneous Transport and Adsorption

Percutaneous absorption of chemicals for therapeutic benefit has always been the basis for topical treatments in dermatology. More recently, the use of this method of administration has gained additional interest with the development of transdermal technology to provide an alternative to traditional intravenous (iv) or oral routes of administration.

Percutaneous absorption has a number of applications not the least being to treat the exterior skin, underlying structures (e.g. structures surrounding a joint) or to provide alternative routes to achieve systemic concentrations of target compounds.

The healthy skin is an impermeable barrier to the loss of hydration from within the body and invasion of foreign material from external sources. Developing treatments for external application must reflect the desired functional rationale for the treatment (i.e. skin surface application, underlying structures or systemic targets). Each requires different functional components to help permeate the relevant structures in the skin.

Percutaneous absorption refers to the absorption of topical medications through the epidermal barrier into underlying tissues and structures with transfer into the systemic circulation. The outermost layer of the epidermis, the stratum cornea, forms the important barrier that regulates the amount and rate of percutaneous absorption.

The formation of this barrier is accomplished through the intercellular lipids along with corneocytes; the primary cell of the epidermis. The lipids comprise free fatty acids, ceramides, as well as cholesterol and are deposited in the intercellular spaces within the stratum corneum. The intercellular lipids provide the primary barrier to molecular movement across the stratum corneum by allowing diffusion at a rate 1,000-fold less than is allowed by cellular membrane.

Corneocytes are cells that have differentiated into structures that contain primarily proteins and only 15% to 30% water. In comparison, other living cells contain approximately 80% to 90% water. The dry corneocytes and hydrophobic intercellular lipids comprise a highly organized and differentiated structure that forms an effective barrier to passage of substances to underlying tissues.

Percutaneous absorption of topically applied medications is accomplished by the process of passive diffusion. It requires substances to pass through the stratum comeum and epidermis, diffuse into the dermis, and eventually transfer into the systemic circulation. Diffusion occurs down a concentration gradient resulting in the dilution of compounds as they progress along the gradient. In addition, the compound may be bound or metabolised as it passes through the underlying tissues. All of these factors will affect the potency of the medication, the level of systemic absorption, and ultimately its efficacy.

Topically applied medication therefore must be developed with the correct components to provide adequate penetration for the required use. Most topically applied substances, particularly nonpolar or hydrophobic compounds, are absorbed by diffusion across the stratum corneum and epidermis through the intercellular corridors. However, polar or hydrophilic substances are transported through the transcellular absorption route. Hair follicles and eccrine sweat ducts may also serve as diffusion shunts for certain substances such as ions, polar compounds, and very large molecules that would otherwise move through the stratum corneum very slowly because of their high molecular weight.

Skin characteristics are an essential consideration for percutaneous absorption. Features of normal skin, barrier changes in the skin, and vascular changes in the skin all play a critical role in absorption.

One of the most important factors affecting percutaneous absorption is skin hydration and environmental humidity. In the normal state of skin hydration, the stratum corneum may be penetrated only by medications passing through the tight, relatively dry, lipid barrier between cells. However, when the skin is hydrated, water molecules bind to hydrophilic lipids between the corneocytes and enable water-soluble medications to more easily diffuse. Therefore, absorption of topical therapies is enhanced by hydration of the skin.

Several additional characteristics of the skin can affect percutaneous absorption of an applied medication. Increased cutaneous vasculature or vasodilatation at the site of application which frequently occurs with inflammation can enhance both local and systemic effects of the drug. This, along with increased surface area of the drug application, will boost overall percutaneous absorption.

The rate-limiting factor of percutaneous absorption seems to be diffusion through the stratum corneum and hence the effectiveness of the epidermal permeability barrier correlates inversely with percutaneous absorption.

Therefore, to increase the efficiency of diffusion into and beyond the stratum comeum, a penetration enhancer can be included in the formulation of the topically applied medication. This material increases the rate of diffusion into the tissues so enhancing the therapeutic effect by increasing the percutaneous concentration of active material, or achieving the same rate of diffusion with a lower initial concentration of topically applied material.

Delivery is an important issue in the development of any drug product, and the choice of a delivery route is contingent upon optimising drug delivery while maintaining convenience and ease of administration.

Transdermal drug delivery provides excellent control of the rate of delivery directly into the bloodstream. It also offers a predictable pharmacokinetic profile and constant drug levels over extended periods of time without the extreme peak/trough fluctuations inherent in oral administration.

Transdermal patches offer benefits similar to those of oral administration in that both are easy for patients to self-administer and place few restrictions on patients' daily activities. Transdermal drug delivery offers the best of IV and oral administration

### DESCRIPTION OF THE INVENTION

The combined use of anti-inflammatory and anti-allergy components offers beneficial therapeutic opportunities over treatment with a single component. The use of omega-3 series PUFAs, particularly the eicosanoid and tetraenoic acids, formulated with flavonoids offers a combination treatment with great potential for the treatment of asthmatic and arthritic disease.

The invention provides pharmaceutical or veterinary composition comprising a flavonoid or a pharmaceutically or veterinarily acceptable derivative thereof, such as a glucuronide, together with an extract of polyunsaturated fatty acids derived from Perna canaliculus, said fatty acids including at least one ω-3 eicosanoid fatty acid. The eicosanoid fatty acid and other fatty acids may be present as free fatty acid, or as a triglyceride, diglyceride, methyl, ethanoic or other ester or a salt. Di- or tri- glycerides may be mixed glycerides in which different fatty acids are present.

The flavonoid may be a flavonol, a flavone, a flavanone, a flavan-3-ol, or an anthocyanidin. Specifically it may be quercetin, rutin, kaempferol, myricetin, isorhamnetin, apigenin, luteolin, hesperetin, naringenin, eriodictyol, a catechin, an epicatechin, a theaflavin, a thearubigin, cyanidin, delphinidin, malvidin, pelargonidin, peonidin, or petunidin. Mixtures of any two or more of these or other flavonoids may be used.

Said at least one ω-3 eicosanoid fatty acid is preferably ω-3 eicosatetraenoic acid.

In an alternative aspect, the invention provides a pharmaceutical or veterinary composition comprising luteolin or a pharmaceutically or veterinarily acceptable derivative thereof, such as a glucuronide, together with ω-3 eicosanoid or omega-3 tetraenoic fatty acid. The eicosanoid or tetraenoic fatty acid and other fatty acids may be present as free fatty acid, or as a triglyceride, diglyceride, methyl, ethanoic or other ester or a salt. In such a composition, the ω-3 eicosatetraenoic acid or tetraenoic acid is preferably present as a component of an extract of polyunsaturated fatty acids derived from Perna canaliculus.

According to either aspect of the invention, compositions of the invention are suitably for oral administration. Such compositions may again comprise a pharmaceutically or veterinarily acceptable diluent or carrier. Suitable examples include water, preferably sterile, or a vegetable oil. Such compositions may be formulated as a syrup, solution, capsule, lozenge, chewable soft tablet, rapid dissolving wafer or the composition can be adsorbed to an inert powder, such as lactose, thereby facilitating a subsequent standard tableting process. For rectal administration a suppository format may be employed.

The composition may be in unit dosage form, wherein each unit dosage form suitably contains from 5 to 200 mg of flavonoid or said derivative thereof or from 10 to 100 mg or from 20 to 50 mg. Such a composition in unit dosage form may be such that each unit dosage form contains from 5 to 500 mg of eicosanoid or tetraenoic fatty acid or said derivative therefor or from 50 to 300 mg or from 100 to 200 mg.

Liquid dosage forms may be put up in unit dose format, e.g. in sachets of a single dose or may be presented in multiple dose format, e.g. in a bottle containing several or many doses. Compositions in liquid dosage form may suitably contain a concentration of 0.5 to 25% (w/v) of flavonoid or said derivative therefor or from 1 to 20% (w/v) or from 5 to 15% (w/v. Such a composition in unit dosage form may be such that each unit dosage form contains from 0.5 to 25% (w/v) of eicosanoid or tetraenoic fatty acid or said derivative therefor or from 1 to 20% (w/v) or from 5 to 15% (w/v).

Oral formulations of the invention may be presented as food or feed supplements or for addition to drinking water.

In all of these compositions, the weight ratio of said flavonoid or derivative thereof to said eicosanoid or tetraenoic acid fatty acid or derivative thereof is from 1:1 to 1:100, e.g. any of 1:4, 1:5, 1:10 and 1:50. Where a mixture of fatty acids extracted from Perna canaliculus is present, the weight ratio of flavonoid to total extracted fatty acids is preferably from 1:1 to 1:100, e.g. 1:5, 1:10 or 1:50.

For the reasons explained above, said eicosanoid or tetraenoic fatty acid or derivative thereof is preferably provided as an extract of fatty acids from Perna canaliculus. This may be an unselected extract of fatty acids from Perna canaliculus or may be especially enriched in the eicosanoid or tetraenoic fatty acids either through purification from a starting extract or by the choice of extraction conditions being such as to favour the extraction of the eicosanoid fatty acids with respect to non-eicosanoid fatty acids. In particular, it is preferred according to the second aspect of the invention that the eicosanoid fatty acid is or comprises ω-3 eicosatetraenoic acid. According to each aspect of the invention, Ω-3 eicosatetraenoic acid preferably constitutes at least 0.05 (w/w) of the fatty acid content of the composition or from 0.05 to 3% (w/w) or from 0.1 to 1.0% (w/w).

Hyaluronic acid (HA) is a high molecular weight glycosaminoglycan, or GAG, which plays a vital role in the functioning of extracellular matrices. HA is also important in that it has numerous actions in the mechanisms associated with inflammation and the wound healing process.

HA is a polymer of glucuronic acid and N-acetylglycosamine, bonded alternatively by glycosidic beta (1,3) and beta (1,4) bonds (FIG. 3). Hyaluronic acid interacts with other proteoglycans and collagen to give stability and elasticity to the extracellular matrix of connective tissue and has essential physico-chemical properties vital to healthy periodontal tissue.

Hyaluronic acid binds to different proteins and water molecules by means of hydrogen bonds to form a viscous macroaggregate whose primary function is to regulate the hydration of tissues, the passage of substances in the interstitial compartment and the structure of connective tissue extracellular matrix. Hyaluronic acid is highly viscous and is found in a wide variety of body tissues, e.g. Vitreous humour of the eye, synovial fluid, umbilical cord, cartilaginous tissue, synovium, the skin, the mucosa of the oral cavity. The polymer can bind up to 50 times its own weight of water and associates with specific proteins and tissue components. HA forms a viscous cement, regulates the water content of the tissue, controls the movement of substances (nutrients, toxins, etc.) into the extracellular spaces and prevents the formation of oedemas which occur on tissue inflammation or injury.

In addition, hyaluronic acid bind to cellular receptors that are expressed only in cells in active division, it also acts as a regulator of migration and cellular division mechanisms which are especially important in healing and tissue repair.

Normal joint structure consists of two adjoining bones capped with cartilage and sealed by the synovial membrane, which itself encloses synovial fluid that acts as a cushion to dampen the compressive forces occurring when the joint is compressed. Synovial fluid also has various physiological functions providing for a healthy cartilage and synovial membrane.

Cartilage is a form of specialised connective tissue designed to be tough and flexible. It is composed of extracellular matrix with embedded protein collagenous structures to give it tensile strength but retaining a smooth physical surface.

The extracellular matrix is a complex structure consisting of various polymers of amino sugars and sugar molecules in long glycosaminoglycan chains binding to proteins to form a mesh of supportive structures; the proteoglycans.

GAGs also include glucosamine and chondroitin. The link between proteoglycans and collagens that underlie the structure of cartilage is hyaluronic acid.

Without HA the cartilage structure breaks down and this is typically seen when subchondral bones are exposed in arthritis producing catabolic enzymes that hydrolyse HA to shorter chain lengths. As the extracellular cement unravels its structure more GAGs are lost and hydrolysed. Indeed there is an inverse correlation between the severity of arthritis and loss of GAGs in a joint.

Clinically, there are three requirements for the management of arthritis:

1. Control inflammation and therefore pain

2. Maintain mobility

3. Reduce joint degeneration, or its progress.

HA is the most important GAG present in connective tissue, such as joint cartilage. It is required to form 50% of the synovial fluid as well as linking protein to proteoglycans, so acting as the “backbone” of connective tissue structure.

Historically, HA has been administered by orthopaedic surgeons as intra-articular injection directly into the joint for the treatment of arthritis and had clinical uses in veterinary as well as human medicine. It is also used in ophthalmology, burn dressings and dermatology, particularly wound healing, implant technology and surgery to prevent adhesions.

Compositions of the present invention may further comprise a glycosaminoglycan such as a hyaluronic acid or a salt thereof or an ester of hyaluronic acid with an alcohol of the aliphatic, heterocyclic or cycloaliphatic series, or a sulphated form of hyaluronic acid, or a glucosamine salt such as a hydrochloride or sulphate, chondroitin 4 or 6 sulphates, dermatan sulphate or keratan sulphate.

Where a composition of the invention is for topical administration, it preferably comprises a pharmaceutically or veterinarily acceptable diluent or carrier. Such a diluent may be water, preferably sterile water, or may be organic solvent, or vegetable oil-based. It may contain skin penetrate ingredients serving to speed penetration of the skin by the active ingredients. These include for instance methanol or non-ionic surfactants or ionic surfactants or mixtures of these. The compositions may comprise stabilising ingredients such as anti-oxidants, suitable anti-oxidants include vitamin C (ascorbic acid), or vitamin E (alpha tocopherol). The composition may also include salts to buffer the solution to physiological pH.

Topical formulations may be formulated as a cream, ointment, lotion, poultice or gel, or they may be incorporated into a patch to be applied to the skin, the patch may have a single or multilayer constructions.

Preferred compositions, especially topical compositions, may contain a concentration of glycosaminoglycan such as hyaluronic acid or a said derivative thereof in an amount of from 1 to 20% (w/w) or from 5 to 15% (w/w) or from 10 to 20% (w/w) based on the total weight of the composition.

The composition may be in unit dosage form, wherein each unit dosage form contains from 5 to 500 mg or from 10 to 250 mg or from 20 to 50 mg of hyaluronic acid or said derivative thereof. Such a composition in unit dosage form may be such that each unit dosage form contains from 5 to 500 mg or from 10 to 250 mg or from 20 to 50 mg of said eicosanoid or tetraenoic fatty acid or derivative thereof.

Liquid dosage forms may be put in unit dose format, e.g. in sachets of a single dose or may be presented in multiple dose format, e.g. in a bottle containing several or many doses. Compositions in liquid dose form may suitable contain a concentration of from 1 to 20% (w/v) of hyaluronic acid or said derivative thereof or from 5 to 15% (v/v) or from 10 to 15% (v/v). They may contain a concentration of from 1 to 20% (w/v) of said eicosanoid or tetraenoic fatty acid or said derivative thereof or from 5 to 15% (v/v) or from 10 to 15% (v/v).

In all of these compositions, the weight ratio of said hyaluronic acid or derivative thereof to said eicosanoid or tetraenoic fatty acid or derivative thereof is from 1 to 1, 1 to 5, 1 to 10, up to 1 to 100.

A number of forms of hyaluronic acids are available from various sources. These include natural sources such as cockerel combs or other animal connective tissue sources and also from bacterial sources such as Streptococcus zoepidicus. The molecular weights of hyaluronic acids range from 50,000 upwards to about 8×10⁶ Daltons. We prefer that said hyaluronic acid or derivative thereof is a low molecular weight form, having a molecular weight of from 50,000 to 500,000, more preferably, having a molecular weight of from 150,000 to 250,000, e.g. about 200,000.

As mentioned above, menthol is preferred as a percutaneous enhancer and promoter of increased transdermal flux of polyunsaturated fatty acids (PUFAs), and glycosaminoglycans and specifically hyaluronic acid alone or in combinations, in the treatment of for instance arthritis, asthma, chronic obstructive pulmonary disease cystic fibrosis, eczema, psoriasis or any other applicable or related conditions.

Topical preparations of PUFAs by their physical nature and characteristics will permeate the lipid-rich intercellular area of the stratum comeum. However, this has been found to be chain-length dependent (Drug Development and Industrial Pharmacy (1999), 25(11), 1209-1213).

Therefore the addition of menthol in concentrations of 0.1% to 20 by weight (more preferably 0.1 to 10%, (e.g. 1 to 5%) in a suitable carrier to a mixture containing one or more polyunsaturated fatty acids, either omega-3 or omega-6 series, will enhance the percutaneous flux of PUFAs into subcutaneous tissues and systemic circulation. Additionally, other compounds in the topical applications will have improved flux when incorporated into a system containing menthol.

Such menthol containing formulations for percutaneous application to the skin and applicable to treat conditions such as localised inflammation and swelling associated with arthritis of the knees, elbows, shoulders, etc. or any joint. Presented as a cream, lotion or gel they allow percutaneous absorption of the components to the underlying structures such as synovial membranes and capsular tissues.

Transdermal application presents an alternative delivery method of oral application for any of the presentations above and specifically for application in asthmatics, arthritics to achieve systemic concentrations sufficient to achieve therapeutic effect. Transdermal compositions may be presented as a single or multi-layered system of therapeutic components and menthol as a percutaneous enhancer or as reservoir-based systems where the mixture with menthol is held in a reservoir and released over time through permeable membranes on to the skin. Alternatively, an adhesive-based system can be used where the components, with menthol, are added to the adhesive layer where they permeate the skin.

The invention includes a method of therapy comprising administering to a mammal suffering from an asthmatic condition, chronic pulmonary obstructive disease, an arthritis condition or other inflammatory condition an effective amount of a flavonoid or pharmaceutically or veterinarily acceptable derivative thereof such as a glucuronide and of at least one polyunsaturated fatty acid or derivative thereof such as a methyl ester, ethanoic ester or a salt, preferably an extract of polyunsaturated fatty acids derived from Perna canaliculus, said fatty acids including at least one ω-3 eicosanoid fatty acid or a tetraenoic fatty acid, separately or as an admixture.

In accordance with the second aspect of the invention, there is included a method of therapy comprising administering to a mammal suffering from asthmatic condition, chronic pulmonary obstructive disease, an arthritis condition or other inflammatory condition an effective amount of luteolin or pharmaceutically or veterinarily acceptable derivative thereof and of an ω-3 eicosanoid or tetraenoic fatty acid, separately or as an admixture.

Such therapeutic methods can of course be advantageously practised using any of the compositions of the invention described herein. Suitable dosages for the flavonoid component are from 0.1 to 100 mg/kg body weight per day or from 1 to 10 mg/kg body weight and suitable dosage amounts for the ω-3 eicosanoid fatty acid component are from 1 to 500 mg/kg body weight per day or from 2 to 100 mg/kg body weight

As used in the preferred practice of the invention, the most potent and naturally occurring anti-inflammatory omega-3 series PUFAs discovered to date are those present in the lipid extracts of New Zealand Green Lipped Mussel, Perna canaliculus, including the 18 and 20-carbon tetraenoic acids. These act upon inflammatory white cells to inhibit cyclo-oxygenase and lipoxygenase activity in vitro and in vivo, with particular activity as anti-leukotrienes.

Of the flavonoids, a number exhibit potency as TNFα inhibitors and are therefore candidates for inhibiting the IgE-mediated histamine release from mast cells that stimulates the inflammatory process in the lungs. They also demonstrate some anti-lipoxygenase activity and so have favourable overlapping anti-inflammatory profiles with lipid extracts from Perna canaliculus. In particular, luteolin demonstrates the most potent anti-TNFα and anti-inflammatory activity of the flavonoids and the formulation of a treatment for asthma, chronic obstructive pulmonary disease or osteo or rheumatoid arthritis produces a pronounced and unexpected synergistic therapeutic effect, effective in all mammalian species.

In summary there are substantial and surprising benefits from combining the anti-inflammatory action, anti-allergic activity of luteolin and the profound effect of the potent anti-leukotriene activity associated with the Perna canaliculus lipid extract.

### EXAMPLES

### Example 1

Investigating the effects of supplementing primary cultures of equine and human monocytes with Green Lipped Mussel lipid extracts and luteolin on the inflammatory mediators produced by the lipoxygenase and cyclo-oxygenase pathways and on the production of TNFα.

Peripheral blood mononuclear cells (monocytes) are prepared from equine and human blood by centrifugation and ficol gradient techniques and cultured in vitro.

Cell cultures are challenged with LPS (bacterial endotoxin), to mimic in vivo challenges, and production of prostaglandins (PGE₂) and leukotrienes (LTB₄ and 5-HETE) and TNFα are measured using ELISA and HPLC techniques.

A range of concentrations of Green Lipped Mussel lipid extracts and luteolin are incubated with LPS-stimulated monocytes and the IC₅₀ determined for each relevant pathway.

### Example 2

A Double-Blind Placebo Controlled Clinical Trial comparing the Efficacy of the Green Lipped Mussel Lipid Extract and Flavonoid alone and in Combination on Recurrent Airway Obstruction in Horses.

Recurrent airway obstruction (RAO), an equine asthma, is a chronic inflammatory disease of the airways typified by bronchoconstriction, wheezing and increased mucous production and caused by allergy to fungal and other dusts but mediated, in part, by leukotrienes and other lipid mediators. The aetiology of RAO and asthma shows multiple possible origins typically associated with allergic and inflammatory components. The purpose of the study was to assess the efficacy of a combined treatment incorporating the anti-inflammatory lipid extract from New Zealand green-lipped mussel (GLM), Perna canaliculus and the anti-allergic compound Luteolin to confirm that the combination was superior in efficacy to the individual components. Studies included clinical status, exercise recovery, breath condensate, broncho-alveolar lavage, cytology and mucous scorings.

Subjects

Eight horses aged 9-21 with RAO symptoms and cytology consistent with the accepted definition of RAO were recruited.

Study Design

The study was a double blind randomized, placebo controlled trial. Qualifying horses were initially examined and randomly assigned to receive the placebo, or the GLM, or the luteolin, or a mixture of GLM and luteolin in a chalk carrier. Randomization was computer generated in balanced blocks of the four treatment regimes. No other medications were administered. Dosage was 4 capsules per day, each containing 50 mg GLM, or 12.5 mg luteolin, or 50 mg GLM plus 12.5 mg luteolin, or being placebo.

Measurements

Subjects were clinically and endoscopically examined and BAL and condensate collected and frozen in liquid nitrogen every two weeks. Each treatment was administered for 28 days. Expired breath condensate was collected using a mask and condensing device cooled by crushed ice. The BAL cytology was scored by neutrophil count. Mucous was scored by location, type, volume and viscosity.

Adverse Reactions

No adverse reactions were observed or reported.

Analysis of Data

A General Linear model of Asthma versus treatment analysis of variance for asthma using adjusted SS scores.

Results

The clinical assessments scores were assessed as a mean clinical RAO score. The clinical assessments were evaluated on a scale of 1-10 where 1 was severe disease symptomology and 10 indicated a disease-free condition score and the results are shown in Table 1 and FIG. 3. The individual data obtained for the eight horses entered into the respiratory study and the statistical analysis of the data is shown below. Supplementing the horses with either GLM lipids or Luteolin significantly (P<0.001) improved the respiratory function. These compounds were shown to have a synergistic effect; with further enhancement of at least 25% when these two substances were administered together (p<0.001). Table 1

| | Mean | SEM |
|---|---|---|
| GLM Lipids | 6.57^{a} | 0.26 |
| Luteolin | 3.38^{ade} | 0.23 |
| Bio-active Lipid + Luteolin | 8.61^{bdf} | 0.19 |
| Inert Carrier | 0.19^{cef} | 0.16 |

Values are presented as Mean±SEM. Values in columns with the same superscript differ significantly: ^{abcdefg}P<0.001

What is claimed is:

1. A pharmaceutical or veterinary composition comprising a flavonoid or a pharmaceutically or veterinarily acceptable derivative thereof together with an extract of polyunsaturated fatty acids derived from Perna canaliculus, said fatty acids including at least one ω-3 eicosanoid fatty acid.

2. A composition as claimed in claim 1, wherein the flavonoid is a flavonol, a flavone, a flavanone, a flavan-3-ol, or an anthocyanidin.

3. A composition as claimed in claim 2, wherein the flavonoid is quercetin, rutin, kaempferol, myricetin, isorhamnetin, apigenin, luteolin, hesperetin, naringenin, eriodictyol, a catechin, an epicatechin, a theaflavin, a thearubigin, cyanidin, delphinidin, malvidin, pelargonidin, peonidin, or petunidin.

4. A composition as claimed in claim 1, wherein said at least one ω-3 eicosanoid fatty acid is ω-3 eicosatetraenoic acid.

5. A pharmaceutical or veterinary composition comprising luteolin or a pharmaceutically or veterinarily acceptable derivative thereof together with an ω-3 eicosanoid or ω-3 tetraenoic fatty acid or ester or salt thereof.

6. A composition as claimed in claim 5, wherein said ω-3 eicosatetraenoic acid is present as a component of an extract of polyunsaturated fatty acids derived from Perna canaliculus.

7. A composition as claimed in claim 1, further comprising a hyaluronic acid or a salt thereof or an ester of hyaluronic acid with an alcohol of the aliphatic, heterocyclic or cycloaliphatic series, or a sulphated form of hyaluronic acid.

8. A composition as claimed in claim 1, for oral or rectal administration.

9. A composition as claimed in claim 8, further comprising a pharmaceutically or veterinarily acceptable diluent or carrier.

10. A composition as claimed in claim 9, formulated as a syrup, solution, capsule, lozenge, tablet, chewable soft tablet, or dissolving wafer.

11. A composition as claimed in claim 10, in unit dosage form, wherein each unit dosage form contains from 5 to 200 mg of flavonoid or said derivative thereof.

12. A composition as claimed in claim 11, wherein each unit dosage form contains from 5 to 500 mg of said eicosanoid fatty acid or derivative thereof.

13. A composition as claimed in claim 9, in liquid dosage form, wherein composition contains a concentration of from 0.5 to 25 wt % of flavonoid or said derivative thereof.

14. A composition as claimed in claim 13, composition contains a concentration of from 0.5 to 25 wt % of said eicosanoid fatty acid or said derivative thereof.

15. A composition as claimed in claim 1, formulated for topical application.

16. A composition as claimed in claim 15, further comprising a pharmaceutically or veterinarily acceptable diluent or carrier.

17. A composition as claimed in claim 15, formulated as a cream, ointment, lotion, poultice or gel or skin patch.

18. A composition as claimed in claim 15, further comprising one or more skin penetration agents.

19. A composition as claimed in claim 18, comprising menthol as a skin penetration agent.

20. A composition as claimed in claim 19, wherein menthol is present at a concentration of from 0.1 to 20 wt %.

21. A composition as claimed in claim 1, wherein the weight ratio of said flavonoid or derivative thereof to said eicosanoid fatty acid or derivative thereof is from 1:1 to 1:100.

22. A composition as claimed in claim 1, wherein said eicosanoid fatty acid or derivative thereof is provided as an extract of fatty acids from Perna canaliculus and said composition contains a concentration of from 1:1 to 1:100 of said extract.

23. A composition as claimed in claim 22, wherein ω-3 eicosatetraenoic acid constitutes at least 0.05 wt % of the fatty acid content of the composition.

24. A method of therapy comprising administering to a mammal suffering from an asthmatic condition, chronic pulmonary obstructive disease, an arthritis condition or other inflammatory condition an effective amount of a flavonoid or pharmaceutically or veterinarily acceptable derivative thereof and one or more polyunsaturated fatty acids, or a salt or ester thereof, separately or as an admixture.

25. A method as claimed in claim 24, wherein the poly unsaturated fatty acids are an extract of derived from Perna canaliculus, said fatty acids including at least one ω-3 eicosanoid fatty acid or a tetracnoic fatty acid.

26. A method of therapy comprising administering to a mammal suffering from an asthmatic condition, chronic pulmonary obstructive disease, an arthritis condition or other inflammatory condition an effective amount of luteolin or a pharmaceutically or veterinarily acceptable derivative thereof and of an ω-3 eicosanoid fatty acid, separately or as an admixture.

27. (canceled)

28. (canceled)

29. (canceled)

30. (canceled)
